(19) United States
(12) Patent Application Publication
(10) Pub. No.: US 2025/0368894 A1
(43) Pub. Date: Dec. 4, 2025

(54) ALGAL-BASED CORROSION INHIBITOR COMPOSITIONS AND METHODS THEREOF

(71) Applicant: SAUDI ARABIAN OIL COMPANY, Dhahran (SA)

(72) Inventor: Harry Daniel ODURO, Dhahran (SA)

(73) Assignee: SAUDI ARABIAN OIL COMPANY, Dhahran (SA)

(21) Appl. No.: 18/679,947

(22) Filed: May 31, 2024

(51) Int. Cl. C09K 15/24; C07C 231/02; C07C 233/27

(52) U.S. Cl. CPC C09K 15/24; C07C 231/02; C07C 233/27

(57) ABSTRACT

Methods for using algal-based corrosion inhibitor compositions may comprise providing a corrosion inhibitor composition comprising an amidation reaction product of a dopamine and a triglyceride; contacting a metal surface with the corrosion inhibitor composition; and allowing the corrosion inhibitor composition to interact with the metal surface so as to inhibit corrosion of the metal surface.

### FIELD OF THE DISCLOSURE

The present disclosure relates generally to corrosion inhibitors and, more particularly, to algal-based corrosion inhibitors useful for inhibiting metal corrosion.

### BACKGROUND OF THE DISCLOSURE

Carbon and iron steel alloys are prevalently utilized in the conveyance of crude oil and natural gas, attributed to the alloys' remarkable ductility, superior ability to absorb stress, and cost-efficiency. Despite these advantages, the operation integrity of steel pipelines may commonly be compromised by corrosion, a degradation process accelerated by the corrosive nature of hydrocarbons. This corrosion is influenced by various factors, including pressure, temperature, pH levels, and the presence of dissolved CO₂, H₂S, and formation water. These factors collectively diminish the structural integrity and lifespan of the alloy materials employed in the infrastructure.

The process of corrosion in subterranean pipelines, particularly exacerbated by acid treatments, is a significant concern in the oil and gas sector. Acidizing processes, designed to enhance the permeability of reservoir rocks, can introduce highly corrosive environments within the geological formations. These treatments often involve the injection of acid solutions, such as hydrochloric acid, into the wellbore to dissolve sediments and stimulate fluid flow. However, the interaction between these acid solutions and the metal components of the infrastructure can lead to accelerated corrosion rates. This corrosion not only affects the pipe's material integrity but also increases the susceptibility of the pipeline to failures and leaks, necessitating more frequent inspections, maintenance, and replacements.

The implications of pipeline corrosion extend beyond the immediate physical damage to include significant economic burdens on the hydrocarbon industry. These burdens manifest as heightened maintenance and operational expenditures (OPEX), periodic shutdowns resulting in production losses, and the need for extensive replacement and overhauling of pipelines, thereby inflating capital expenditures (CAPEX). Recent assessments estimate the global annual cost of corrosion within the oil and gas production industry at approximately 1.4 billion USD. This figure comprises 589 million USD allocated for surface pipeline and facility costs, 463 million USD for downhole tubing expenses, and an additional 320 million USD directed towards capital expenditures related to corrosion management. The industry acknowledges that effective corrosion management strategies not only facilitate cost reduction but also align with safety, health, and environmental compliance standards.

In the context of oil and gas production fields, CO₂ and H₂S emanating from carbonate and sour reservoirs are present in significant concentrations within the gas and aqueous phases. These compounds can induce acidity through thermochemical sulfate reduction processes yielding H₂S or stimulate bio-corrosion, in which microorganisms such as sulfur-reducing bacteria generate sulfur species, fostering sour environments and biofilms. These biological and chemical mechanisms contribute to the corrosion of carbon and iron steel surfaces under high-temperature, high-pressure conditions. To mitigate these effects, various prevention processes have been adopted, including the employment of corrosion control chelating agents, dilution or caustic washing to lower the total acid number in heavy crude oils, metal alloying, electroplating, and the development of metal oxide layers on metallic surfaces, alongside the use of corrosion inhibitors. Nonetheless, these strategies are not devoid of limitations, often constrained by their efficacy, availability, and associated costs.

The deployment of numerous organic and inorganic compounds as corrosion inhibitors in the oil and gas sectors aims to address these challenges. Despite their potential, the application of such inhibitors, particularly in environments characterized by the presence of sour petroleum, is restricted by limited availability, concerns over long-term corrosion efficiency, and performance-related costs. This highlights the ongoing need for the development of corrosion mitigation technologies, aimed at enhancing the durability and reliability of pipeline infrastructure in the face of complex subterranean conditions.

### SUMMARY OF THE DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, methods for using algal-based corrosion inhibitor compositions may comprise providing a corrosion inhibitor composition comprising an amidation reaction product of a dopamine and a triglyceride; contacting a metal surface with the corrosion inhibitor composition; and allowing the corrosion inhibitor composition to interact with the metal surface so as to inhibit corrosion of the metal surface.

In another embodiment, methods for preparing algal-based corrosion inhibitor compositions may comprise providing a dopamine and a triglyceride; interacting the dopamine with the triglyceride at a set of reaction conditions; wherein a molar ratio of the dopamine to the triglyceride is about 1:4 to about 4:1; and obtaining an amidation reaction product.

In a further embodiment, algal-based corrosion inhibitor compositions may comprise an amidation reaction product of a dopamine and a triglyceride.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

Not applicable.

### DETAILED DESCRIPTION

Embodiments in accordance with the present disclosure generally relate to corrosion inhibitors and, more particularly, to algal-based corrosion inhibitors useful for inhibiting metal corrosion. Within the context of hydrocarbon systems, the phenomenon of corrosion in oil and gas pipelines is subject to a multitude of factors, including temperature variations, concentrations of CO₂ and H₂S, water composition, flow dynamics, and the physical state of the steel surfaces. Such conditions may precipitate a marked reduction in production efficiency and pose significant safety risks. Corrosion mechanism, particularly prevalent in oil and gas extraction wells, processing apparatus, and transport pipelines, fundamentally involve: an anodic site, or metallic surface, on which corrosion materializes; a cathode, serving as an electrochemical conductor that remains unaltered by the corrosion process; and an electrolyte, which acts as a corrosive or solvent medium facilitating electron migration from the anode to the cathode.

The broad spectrum of corrosion mechanisms in aqueous-gaseous phase environments (e.g., CO₂—H₂O—C or CO₂—H₂O—H₂S—C systems) on stainless steel interfaces is characterized by electron transfer reactions among a range of species (e.g., H₂S, Sn²⁻, HS⁻, Fe²⁺, S₈, FeS, SO₄²⁻, H₂O, CO₂, Cl⁻, H⁺, Fe₂O₃, and FeOOH). These species can engage in redox reactions, potentially leading to corrosion or degradation on pipeline surfaces within aggressive petroleum contexts. A significant diminution in corrosion rates can substantially enhance facility performance and extend the lifespan of components, culminating in considerable benefits such as lower maintenance expenditures and the uninterrupted transport of oil and gas to consumers.

One example method to mitigate corrosion may entail the application of organic corrosion inhibitors, comprising two crucial elements: a polar end enriched with electrons, owing to heteroatoms like oxygen, nitrogen, and sulfur, capable of adsorbing onto and shielding steel surfaces; and a hydrophobic end or hydrocarbon tail, which effectively repels redox species in the electrolyte responsible for internal corrosion. In the petroleum sector, such organic inhibitors are extensively employed to prevent internal corrosion on carbon (C-steel) and iron (Fe-steel) surfaces.

Despite the efficacy of organic corrosion inhibitors (OCIs) in reducing corrosion rates through the formation of protective mono- or multilayer films on steel surfaces, notable declines in inhibitory efficiency and performance have been observed. These declines are attributed to the deterioration of active polar and hydrophobic functional groups within real hydrocarbon systems, especially under the harsh conditions of acidic sour oilfields with multiphase flow dynamics, fluctuating temperatures and pressures, and variable water content containing dissolved gases such as CO₂, H₂S, O₂, and SO₂. Consequently, the petroleum and chemical industries are increasingly exploring sustainable and eco-technological solutions for the development of innovative, efficient bio-based products that offer zero degradation and no waste streams, thereby revitalizing existing OCIs.

The present disclosure describes an efficient and eco-technological approach for synthesizing corrosion inhibitors by amalgamating extracts from palm oil, rich in free fatty acids, with crude dopamine extracts from marine macroalgae. This synthesis may produce algal-based corrosion inhibitors suitable for applications within the oil and gas sectors. Marine microalgae, or seaweeds, are photosynthetic multicellular organisms known for their rich content of phenolic compounds, containing a broad spectrum of bioactive properties. These properties have found extensive applications across various sectors, including food, health and pharmaceuticals, cosmetics, and agriculture, attributed to their antioxidant, anti-inflammatory, antitumoral, hypocholesterolemic, anticoagulant, antiviral, and antimicrobial properties. Given these bioactive and chemically active ingredients, there exists significant potential for investigating the components of algal plant extracts and developing them into industrial-scale corrosion inhibitors with low toxicity and degradation, as part of a green energy initiative within the oil and gas industries.

The rationale for utilizing marine macroalgae as a source for green or bio-based corrosion inhibitors is twofold: the existence of over 10,000 algal species, categorized based on their photosynthetic pigments and their rapid growth rates in marine environments and free-floating ponds; and their non-competition with food crops for arable land. Consequently, there is potential for augmenting profitability through the development of value-added products or raw materials for the oil and gas sectors.

Given the complexity of real hydrocarbon systems, achieving optimal corrosion inhibition is challenging with a singular component. Therefore, corrosion inhibitors designed to protect downhole and pipeline equipment typically comprise a blend of organic components that, through a synergistic effect, provide enhanced inhibitor efficiency compared to individual components. The present disclosure describes the combination of dopamine (e.g., dopamine extracts from macroalgae) with triglycerides (e.g., fatty acid extracts from palm oil) to synthesize anticorrosive products, to ensure the health, safety, and longevity of metal materials in oil and gas applications (e.g., sour oil and gas fields). This synthesis of two complementary natural products into corrosion inhibitors may offer numerous advantages, including low toxicity, minimal residue production, reduced degradation under high-pressure and high-temperature conditions, simplified processes, and decreased economic costs to the oil and gas industries.

Therefore, non-limiting example algal-based corrosion inhibitor compositions may comprise: an amidation reaction product of a dopamine and a triglyceride.

Furthermore, non-limiting example methods of preparing algal-based corrosion inhibitor compositions may comprise: providing a dopamine and a triglyceride; interacting the dopamine with the triglyceride at a set of reaction conditions; wherein a molar ratio of the dopamine to the triglyceride is about 1:4 to about 4:1; and obtaining an amidation reaction product.

Non-limiting example methods of using algal-based corrosion inhibitor compositions may comprise: providing a corrosion inhibitor composition comprising an amidation reaction product of a dopamine and a triglyceride; contacting a metal surface with the corrosion inhibitor composition; and allowing the corrosion inhibitor composition to interact with the metal surface so as to inhibit corrosion of the metal surface.

As previously mentioned, the corrosion inhibitor compositions of the present disclosure may comprise an amidation reaction product of a dopamine and a triglyceride. At least a portion of the dopamine and/or triglyceride may be derived from an organic source. For example, the dopamine may be derived from one or more macroalgae. Macroalgae, given its availability and abundance, may be an ideal source of bioactive compounds such as dopamine. Any suitable macroalgae may be used to obtain dopamine, including, Archaeplastida, Chlorarachniphytes, Euglenids, Heterokonts, Cryptophyta, Dinoflagellata, Haptophyta, and any combination thereof. Chlorophyta (of Archaeplastida), such as Ulvaria obscura, may be preferably used due to the organisms' abundance of dopamine.

Dopamine extracts may be obtained from macroalgae by any suitable method, such as, preferably, by a solvent-based extraction method. In any embodiment, macroalgae may be introduced to a solvent (e.g., dichloromethane, methanol, or a combination thereof), in which the solvent, over a period of time and at a particular temperature, may extract the polar dopamine molecule from the macroalgae. For example, dopamine extraction of the macroalgae may take place over about 12 hr to 120 hr (or about 12 hr to about 24 hr, or about 12 hr to about 72 hr, or about 24 hr to about 72 hr, or about 24 hr to about 120 hr, or about 72 hr to about 120 hr) and at a temperature of about −25° C. to about 25° C. (or about −25° C. to about 0° C., or about 0° C. to about 25° C.). Following the extraction, the solvent may be subsequently evaporated to at least partially isolate the dopamine. The dopamine extracts may undergo further purification, including filtration, to further isolate the dopamine.

At least a portion of the triglyceride may be obtained from an organic source. Vegetable oils, for example, are an abundant and non-toxic source of naturally occurring triglycerides. The vegetable oil may comprise any suitable oil and may preferably comprise palm oil. Palm oil may comprise numerous fatty acid residues including, but not limited to, myristic acid, palmitic acid, palmitoleic acid, stearic acid, oleic acid, linoleic acid, linolenic acid, arachidic acid, eicosenoic acid, the like, and any combination thereof.

The reaction between the dopamine and the triglyceride may comprise an amidation reaction, forming an amidation reaction product. Scheme I represents a non-limiting example amidation reaction of the present disclosure.

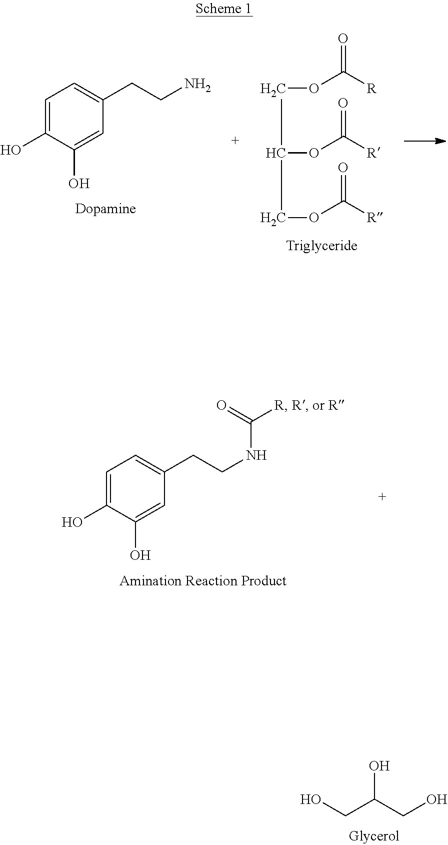

In Scheme I, R, R′, and R″ are optionally unsaturated hydrocarbyls, such as a C₁₀-C₃₀ alkane or a C₁₀-C₃₀ alkene. R, R′, and R″ may each represent different optionally unsaturated hydrocarbyls, or one or more of R, R′, and R″ may represent the same structure, depending on the particular triglyceride. The molar ratio of the dopamine to the triglyceride may, for example, be about 1:4 to about 4:1 (or about 1:4 to about 1:1, or about 1:1 to about 3:1, or about 1:1 to about 2:1, or about 2:1 to about 4:1, or about 2:1 to about 3:1, or about 3:1 to about 4:1). Preferably, the molar ratio of the dopamine to the triglyceride may be about 3:1.

The amidation reaction may take place under a certain set of reaction conditions. For example, the reaction temperature may be about 75° C. to about 150° C. (or about 75° C. to about 110° C., or about 75° C. to about 130° C., or about 110° C. to about 130° C., or about 110° C. to about 150° C.). Furthermore, the reaction may have a residence time, for example, of about 1 hr to about 5 hr (or about 1 hr to about 4 hr, or about 1 hr to about 3 hr, or about 3 hr to about 5 hr, or about 3 hr to about 4 hr, or about 4 hr to about 5 hr).

The amidation reaction product may be used as a component of a corrosion inhibitor composition. Environments in which corrosion inhibitor compositions of the present disclosure may be particularly effective include any downhole application in which the environment is relatively acidic. Such acidity may pose a problem for any metal surfaces present therein. In any embodiment, the corrosion inhibitor compositions of the present disclosure may be used as an additive in an acidic treatment fluid that is placed downhole. Examples of acidic treatment fluids include, but are not limited to, acid well treatment fluids that may comprise hydrochloric acid, acetic acid, formic acid, hydrofluoric acid, glycolic acid, the like, and any combination thereof. As a component of a treatment fluid, the corrosion inhibitor composition may be present at concentrations of about 0.01 wt % to about 10 wt % (or about 0.01 wt % to about 5 wt %, or about 0.01 wt % to about 2 wt %, or about 0.01 wt % to about 1 wt %, or about 1 wt % to about 10 wt %, or about 1 wt % to about 5 wt %, or about 1 wt % to about 2 wt %, or about 2 wt % to about 10 wt %, or about 2 wt % to about 5 wt %, or about 5 wt % to about 10 wt %), based on the total weight of the treatment fluid. The corrosion inhibitor compositions of the present disclosure may be used in subterranean environments that have a bottom hole temperature ranging from about 70° F. to about 500° F. (or about 70° F. to about 300° F., or about 200° F. to about 300° F., or about 300° F. to about 500° F.).

Optionally, the corrosion inhibitor compositions of the present disclosure may comprise a solvent. The solvent may include, but is not limited to, isopropanol, ethanol, tert-butyl alcohol, tripropylene glycol monomethyl ether, the like, and any combination thereof. When present in a corrosion inhibitor composition, the solvent may have a concentration of about 1 vol % to about 50 vol % (or about 1 vol % to about 25 vol %, or about 1 vol % to about 10 vol %, or about 10 vol % to about 50 vol %, or about 10 vol % to about 25 vol %, or about 25 vol % to about 50 vol %), by volume of the corrosion inhibitor composition.

Optionally, the corrosion inhibitor compositions of the present disclosure may comprise a surfactant. The presence of a surfactant may not be essential or required. Without being bound by any theory, a surfactant may aid in the dispersibility of the corrosion inhibitor composition and/or may assist in the plating of the corrosion inhibitor composition on the metal surfaces to be inhibited. A surfactant may aid in achieving a more uniform plating on the metal surface. The surfactant may comprise a cationic surfactant, an anionic surfactant, a nonionic surfactant, a zwitterionic surfactant, or any combination thereof. The presence of a surfactant may be especially advantageous at temperatures above about 250° F. Examples of surfactants suitable for use in the present disclosure include, but are not limited to, dimethyldicocoalkylamine oxide, lauryl alcohol ethoxylate, cocoalkylamine ethoxylate, or mixtures thereof. When used, a surfactant may be present in an amount from about 0.05 wt % to about 10 wt % (or about 0.05 wt % to about 1 wt %, or about 1 wt % to about 10 wt %), by weight of the corrosion inhibitor composition.

The corrosion inhibitor compositions of the present disclosure may optionally include one or more of a variety of well-known additives, such as gel stabilizers, salts, fluid loss control additives, surfactants, solvents, scale inhibitors, catalysts, clay stabilizers, biocides, bactericides, friction reducers, gases, foaming agents, iron control agents, solubilizers, pH adjusting agents (e.g., buffers), the like, and any combination thereof. Those of ordinary skill in the art, with the benefit of this disclosure, may be able to determine the appropriate additives for a particular application.

The metal surfaces to be protected by the corrosion inhibitor compositions of the present disclosure may include any metal surface susceptible to corrosion in an acidic environment including, but not limited to, ferrous metals, low alloy metals (e.g., N-80 Grade), stainless steel (e.g., 13 Cr), copper alloys, brass, nickel alloys, and duplex stainless steel alloys. Such metal surfaces include downhole piping, downhole tools, and the like.

Embodiments disclosed herein include:

A. Methods for using algal-based corrosion inhibitor compositions, the methods comprising: providing a corrosion inhibitor composition comprising an amidation reaction product of a dopamine and a triglyceride; contacting a metal surface with the corrosion inhibitor composition; and allowing the corrosion inhibitor composition to interact with the metal surface so as to inhibit corrosion of the metal surface.

B. Methods for preparing algal-based corrosion inhibitor compositions, the methods comprising: providing a dopamine and a triglyceride; interacting the dopamine with the triglyceride at a set of reaction conditions; wherein a molar ratio of the dopamine to the triglyceride is about 1:4 to about 4:1; and obtaining an amidation reaction product.

C. Algal-based corrosion inhibitor compositions, the compositions comprising: an amidation reaction product of a dopamine and a triglyceride.

Each of embodiments A, B, and C may have one or more of the following additional elements in any combination:

Element 1: wherein at least a portion of the dopamine is derived from a macroalgae.

Element 2: wherein the macroalgae comprises a Chlorophyta.

Element 3: wherein the Chlorophyta comprises an Ulvaria obscura.

Element 4: wherein at least a portion of the triglyceride is derived from a vegetable oil.

Element 5: wherein the vegetable oil comprises a palm oil.

Element 6: wherein the amidation reaction product comprises Formula I;

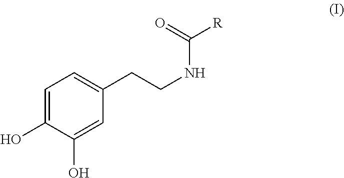

wherein R is an optionally unsaturated hydrocarbyl.

Element 7: wherein the optionally unsaturated hydrocarbyl is a C₁₀-C₃₀ alkane or a C₁₀-C₃₀ alkene.

Element 8: wherein the set of reaction conditions comprises a reaction temperature of about 75° C. to about 150° C. and a residence time of about 1 hour to about 5 hours.

By way of non-limiting example, exemplary combinations applicable to A, B and C include: 1 with 2; 1 with 4; 1 with 6; 1 with 8; 2 with 3; 2 with 4; 2 with 6; 2 with 8; 3 with 4; 3 with 6; 3 with 8; 4 with 5; 4 with 6; 4 with 8; 5 with 6; 5 with 8; 6 with 7; 6 with 8; 7 with 8; 1 with 2 and 4; 1 with 4 and 6; and 1 with 6 and 8.

The present disclosure is further directed to the following non-limiting clauses:
Clause 1. A method comprising:
   providing a corrosion inhibitor composition comprising an amidation reaction product of a dopamine and a triglyceride;
   contacting a metal surface with the corrosion inhibitor composition; and
   allowing the corrosion inhibitor composition to interact with the metal surface so as to inhibit corrosion of the metal surface.
Clause 2. The method of clause 1, wherein at least a portion of the dopamine is derived from a macroalgae.
Clause 3. The method of clause 2, wherein the macroalgae comprises a Chlorophyta.
Clause 4. The method of clause 3, wherein the Chlorophyta comprises an Ulvaria obscura.
Clause 5. The method of any one of clauses 1-4, wherein at least a portion of the triglyceride is derived from a vegetable oil.
Clause 6. The method of clause 5, wherein the vegetable oil comprises a palm oil.
Clause 7. The method of any one of clauses 1-6, wherein the amidation reaction product comprises Formula (I);

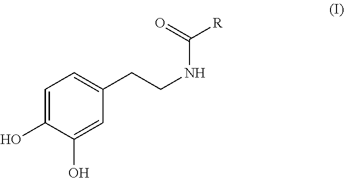

wherein R is an optionally unsaturated hydrocarbyl.
Clause 8. The method of clause 7, wherein the optionally unsaturated hydrocarbyl is a C₁₀-C₃₀ alkane or a C₁₀-C₃₀ alkene.
Clause 9. The method of any one of clauses 1-8, wherein the set of reaction conditions comprises a reaction temperature of about 75° C. to about 150° C. and a residence time of about 1 hour to about 5 hours.
Clause 10. A method comprising:
   providing a dopamine and a triglyceride;
   interacting the dopamine with the triglyceride at a set of reaction conditions;
      wherein a molar ratio of the dopamine to the triglyceride is about 1:4 to about 4:1; and
   obtaining an amidation reaction product.
Clause 11. The method of clause 10, wherein at least a portion of the dopamine is derived from a macroalgae and at least a portion of the triglyceride is derived from a vegetable oil.
Clause 12. The method of clause 11, wherein the macroalgae comprises a Chlorophyta comprising an Ulvaria obscura.
Clause 13. The method of clause 11 or clause 12, wherein the vegetable oil comprises a palm oil.
Clause 14. The method of any one of clauses 10-13, wherein the amidation reaction product comprises Formula (I);

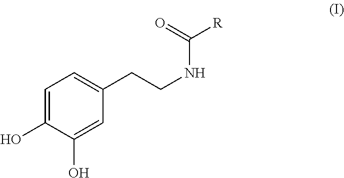

wherein R is an optionally unsaturated hydrocarbyl.
Clause 15. A composition comprising:
   an amidation reaction product of a dopamine and a triglyceride.
Clause 16. The composition of clause 15, wherein at least a portion of the dopamine is derived from a macroalgae.
Clause 17. The composition of clause 16, wherein the macroalgae comprises a Chlorophyta.
Clause 18. The composition of any one of clauses 15-17, wherein at least a portion of the triglyceride is derived from a vegetable oil comprising a palm oil.
Clause 19. The composition of any one of clauses 15-18, wherein the amidation reaction product comprises Formula (I);

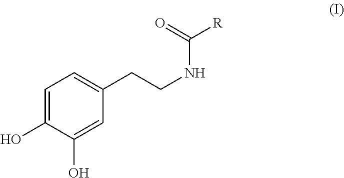

wherein R is an optionally unsaturated hydrocarbyl.
Clause 20. The composition of clause 19, wherein the optionally unsaturated hydrocarbyl is a C₁₀-C₃₀ alkane or a C₁₀-C₃₀ alkene.

### Examples

Dopamine and triglycerides were derived from Ulvaria obscura macroalgae and palm oil, respectively, and utilized to synthesize an amidation reaction product consistent with the present disclosure. The extraction of the dopamine involved harvesting fresh specimens of Ulvaria obscura from Shannon Point Beach, Anacortes, Washington, USA. To facilitate the extraction of crude polar and non-polar compounds, approximately 1.2 kg of the fresh algal biomass was initially dried in an oven at 30° C., before undergoing extraction with 600 mL of a 2:1 dichloromethane:methanol mixture under dark conditions at −7° C. for a duration of 72 hours. Following this, the solvents dichloromethane and methanol were evaporated using a rotary evaporator, yielding the crude dopamine extracts.

The quantification of dopamine within these extracts was performed via high-performance liquid chromatography (HPLC), employing a photodiode array detector at 280 nm wavelength. For analysis, 5 mL aliquots of the extracts were first filtered through GF/A glass fiber filters and subsequently through a 0.22 μm filter. The analysis involved the injection of 10 μL of the filtered extract into the HPLC, employing an isocratic elution process with 0.1 M phosphate buffer (pH 4.0) containing 1.0 mM heptane sulfonic acid and 10% methanol at a flow rate of 1.0 mL/min using a SUPELCOSIL™ LC-ABZ column (5 cm×4.6 mm×5 μm). A standard of dopamine hydrochloride of HPLC grade was utilized for calibration, with the detection limit for dopamine established at 0.9 μM. The calculated dopamine concentration within the Ulvaria obscura extracts was expressed as a weight percent of the fresh biomass, with a value of 65.3 wt % obtained.

The amidation reaction was conducted by directly combining the dopamine extract with triglycerides from West African palm oil (Elaeis guineensis), re-distilled and indigenous, in a 1:2 molar ratio. The process entailed the use of 1.0 L three-necked round-bottom flasks equipped with a heating mantle, a reflux condenser, and a mechanical stirrer, containing 130 mL of triglyceride-rich palm oil (fatty acid residues of C14:0, C16:0, C18:0, C18:1, and C18:2). To this, the 65.3 wt % dopamine was added and the reaction mixture was subjected to vigorous stirring for 3 hours at 110° C., facilitating the conversion of the triglyceride fatty acids and dopamine into the amidation reaction product.

Thin-layer chromatography (TLC) analysis of the resulting amidation reaction product, using a heptane and ethyl acetate solution (9:1, V/V) as the eluent, indicated and 81.2% conversion of triglycerides and 16.9% conversion of dopamine into the amidation reaction product.

The efficacy of the amidation reaction product was assessed within the context of a corrosion inhibitor composition. Three distinct formulations of acidizing treatment fluids were prepared by mixing the corrosion inhibitor with varying concentrations of hydrochloric acid (HCl). The corrosion interaction of these fluids was evaluated on standard C-95 grade stainless steel coupons at a temperature of 285° F., within aging cells maintained at a pressure of 1000 psi over a duration of 6 hours.

The formulations of the acidizing treatment fluids were achieved by combining 90 mL of HCl at concentrations of 5 wt %, 10 wt %, or 20 wt % with 10 mL of a corrosion inhibitor composition. This inhibitor composition comprised a 50:50 (V/V) blend of the aforementioned amidation reaction product and isopropanol. Subsequent to their preparation, four C-95 stainless steel coupons, previously cleaned with distilled water, acetone, and distilled water again, were dried and placed within aging cells filled with the respective acidizing treatment fluids, alongside a control sample containing solely the corrosion inhibitor composition, under a pressure of 1000 psi. Following a reaction period of 6 hours (t), the corrosion rates (C_{R}) of both the acidizing treatment fluids and the control were determined utilizing Equation 1, in which M₀ and M₁ represent the initial and final masses of the stainless steel coupons, respectively, and S denotes the coupon surface area.

The results, summarized in Table 1, indicate that the corrosion rates for all solutions adhered to the industry benchmark of 0.05 lb/ft² for C-95 grade stainless steel. Notably, the occurrence of pitting corrosion was minimal across all acidizing treatment fluids. Significantly, the corrosion rate of the control sample remained well below the threshold of 0.001 lb/ft², underscoring the substantial reduction in stainless steel corrosion rate upon incorporating the amidation reaction product-derived corrosion inhibitor within HCl environments, even under reservoir temperature and pressure conditions. This pronounced reduction in corrosion rates, along with observed efficiency, is possibly attributable to the presence of function groups such as hydroxyl, benzene, amine, and carboxylic acid within the amidation reaction product. These groups are hypothesized to facilitate the formation of a dense and uniform protective layer over the metal surface, thereby effectively mitigating acid-induced corrosion.

TABLE 1

| Composition | Corrosion Rate (lb/ft²) |
|---|---|
| Corrosion Inhibitor | <0.001 |
| Corrosion Inhibitor + 5 wt % HCl | 0.011 |
| Corrosion Inhibitor + 10 wt % HCl | 0.019 |
| Corrosion Inhibitor + 20 wt % HCl | 0.032 |

Sour formation water (comprising 9.2 wt % sulfur, 7.3 wt % H₂S, 5.9 wt % CO₂, with a pH of 4.9) and sour crude oil (comprising 4.9 wt % sulfur, 6.1 wt % H₂S, 4.4 wt % CO₂, with a pH of 5.6) were collected to evaluate the corrosion inhibition capability of the amidation reaction product on carbon steels frequently utilized in environments characteristic of sour petroleum extraction. For this purpose, experimental fluids were formulated by combining 90 mL of either sour water or sour crude oil with 10 mL of the same corrosion inhibitor composition as previously described (50:50 V/V ratio mixture of the amidation reaction product and isopropanol). These experimental fluids were then subjected to aging processes within the same aging chambers again pressurized to 1000 psi, containing either API-5L X65 carbon steel or ASTM A106 carbon steel samples, for a duration of 6 hours. Subsequently, the corrosion rates and the efficiency of corrosion inhibition (Equation 2) were determined. In Equation 2, C_{S} represents the corrosion rates of the carbon steel samples immersed in the experimental fluids, with C_{B} denoting the corrosion rates of the control samples immersed solely in the corrosion inhibitor composition.

The results, as shown in Table 2, reveal that the corrosion rates associated with both the sour water and sour oil experimental fluids, as well as the control samples, adhered to the industry-accepted threshold of 0.05 lb/ft². Furthermore, the examination of carbon steel surfaces did not reveal any conspicuous signs of corrosion, suggesting the potential of the amidation reaction product to form protective adsorption layers on the steel surfaces within sour petroleum media.

TABLE 2

| | API-5L X65 | ASTM A106 |
|---|---|---|
| | Corrosion | Corrosion |
| Composition | Rate (lb/ft²) | Rate (lb/ft²) |
| Corrosion Inhibitor | 0.006 | 0.004 |
| Sour Crude Oil | 0.034 | 0.030 |
| Sour Formation Water | 0.047 | 0.040 |
| Sour Crude Oil + Corrosion Inhibitor | 0.011 | 0.009 |
| Sour Formation Water + Corrosion | 0.012 | 0.010 |
| Inhibitor | | |

Table 3 presents the calculated efficiencies of corrosion inhibition for the experimental fluids in contact with the carbon steel samples. The observed high levels of corrosion inhibition efficiency underscore the effectiveness of the amidation reaction product in mitigating corrosion on carbon steels within sour water and sour crude oil environments. This efficacy is indicative of the amidation reaction product's potential role in forming protective barriers against the corrosive agents present in these challenging environmental conditions.

TABLE 3

| | API-5L X65 | ASTM A106 |
|---|---|---|
| | Corrosion | Corrosion |
| | Inhibition | Inhibition |
| Composition | Efficiency | Efficiency |
| Sour Crude Oil + Corrosion Inhibitor | 82.22% | 85.86% |
| Sour Formation Water + Corrosion Inhibitor | 87.05% | 89.63% |

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains,” “containing,” “includes,” “including,” “comprises,” and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

The invention claimed is:

1. A method comprising:
providing a corrosion inhibitor composition comprising an amidation reaction product of a dopamine and a triglyceride;
contacting a metal surface with the corrosion inhibitor composition; and
allowing the corrosion inhibitor composition to interact with the metal surface so as to inhibit corrosion of the metal surface.

2. The method of claim 1, wherein at least a portion of the dopamine is derived from a macroalgae.

3. The method of claim 2, wherein the macroalgae comprises a Chlorophyta.

4. The method of claim 3, wherein the Chlorophyta comprises an Ulvaria obscura.

5. The method of claim 1, wherein at least a portion of the triglyceride is derived from a vegetable oil.

6. The method of claim 5, wherein the vegetable oil comprises a palm oil.

7. The method of claim 1, wherein the amidation reaction product comprises Formula (I); 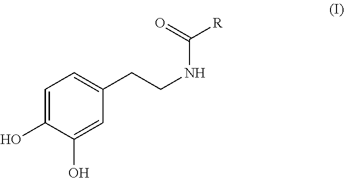
wherein R is an optionally unsaturated hydrocarbyl.

8. The method of claim 7, wherein the optionally unsaturated hydrocarbyl is a C₁₀-C₃₀ alkane or a C₁₀-C₃₀ alkene.

9. The method of claim 1, wherein the set of reaction conditions comprises a reaction temperature of about 75° C. to about 150° C. and a residence time of about 1 hour to about 5 hours.

10. A method comprising:
providing a dopamine and a triglyceride;
interacting the dopamine with the triglyceride at a set of reaction conditions;
wherein a molar ratio of the dopamine to the triglyceride is about 1:4 to about 4:1; and
obtaining an amidation reaction product.

11. The method of claim 10, wherein at least a portion of the dopamine is derived from a macroalgae and at least a portion of the triglyceride is derived from a vegetable oil.

12. The method of claim 11, wherein the macroalgae comprises a Chlorophyta comprising an Ulvaria obscura.

13. The method of claim 11, wherein the vegetable oil comprises a palm oil.

14. The method of claim 10, wherein the amidation reaction product comprises Formula (I); 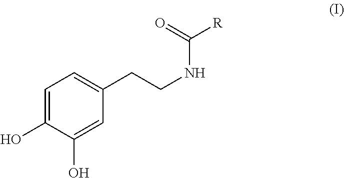
wherein R is an optionally unsaturated hydrocarbyl.

15. A composition comprising:
an amidation reaction product of a dopamine and a triglyceride.

16. The composition of claim 15, wherein at least a portion of the dopamine is derived from a macroalgae.

17. The composition of claim 16, wherein the macroalgae comprises a Chlorophyta.

18. The composition of claim 15, wherein at least a portion of the triglyceride is derived from a vegetable oil comprising a palm oil.

19. The composition of claim 15, wherein the amidation reaction product comprises 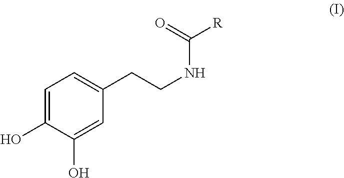
wherein R is an optionally unsaturated hydrocarbyl.

20. The composition of claim 19, wherein the optionally unsaturated hydrocarbyl is a C₁₀-C₃₀ alkane or a C₁₀-C₃₀ alkene.
